(19) United States
(12) Patent Application Publication
(10) Pub. No.: US 2015/0105479 A1
(43) Pub. Date: Apr. 16, 2015

(54) CATALYTICALLY ACTIVE BODY FOR THE SYNTHESIS OF DIMETHYL ETHER FROM SYNTHESIS GAS

(71) Applicant: BASF SE, Ludwigshafen (DE)

(72) Inventors: Alexander Schäfer, Limburgerhog (DE); Mathias Feyen, Hirschberg (DE); Andrei-Nicolae Parvulescu, Heidelberg (DE); Ulrich Müller, Neustadt (DE); Bernd Bastian Schaack, Ludwigshafen (DE)

(21) Appl. No.: 14/396,396

(22) PCT Filed: Apr. 15, 2013
(86) PCT No.: PCT/EP2013/057763

§ 371 Date: Oct. 23, 2014

Related U.S. Application Data
(60) Provisional application No. 61/637,288, filed on Apr. 24, 2012.

(51) Int. Cl. B01J 29/76; C07C 41/09; B01J 29/46

(52) U.S. Cl. CPC B01J 29/76; B01J 29/46; C07C 41/09
USPC 518/713; 502/64

(57) ABSTRACT

The invention relates to a catalytically active body for the synthesis of dimethyl ether from synthesis gas. In particular, the invention relates to an improved catalytically active body for the synthesis of dimethyl ether, whereby the components of the active body comprise a methanol active component and an acid component comprising a zeolitic material being crystallized by means of one or more alkenyltrialkylammonium cation R¹R²R³R⁴N⁺-containing compounds as structure directing agent. Furthermore, the present invention concerns a method for the preparation of a catalytically active body, the use of the catalytically active body and a method for the preparation of dimethyl ether from synthesis gas.

### FIELD OF THE INVENTION

The invention relates to a catalytically active body for the synthesis of dimethyl ether from synthesis gas. In particular, the invention relates to an improved catalytically active body for the synthesis of dimethyl ether, whereby the components of the active body comprise a methanol active component and an acid component comprising a zeolitic material being crystallized by means of one or more alkenyltrialkylammonium cation R¹R²R³R⁴N⁺-containing compounds as structure directing agent. Furthermore, the present invention concerns a method for the preparation of a catalytically active body, the use of the catalytically active body and a method for the preparation of dimethyl ether from synthesis gas.

### BACKGROUND OF THE INVENTION

Hydrocarbons are essential in modern life and used as fuel and raw materials, including the chemical, petrochemical, plastics, and rubber industry. Fossil fuels such as oil and natural gas are composed of hydrocarbons with a specific ratio of carbon to hydrogen. In spite their wide application and high demand, fossil fuels also have limitations and disadvantages in the view of being a finite resource and their contribution to global warming if they are burned.

Research on alternative fuels was mainly started due to ecological and economical considerations. Among the alternative fuels, dimethyl ether (DME), which is recently discovered as a clean fuel, can be synthesized from syngas that was generated from different primary sources. These primary sources can be natural gas, coal, heavy oil and also biomass. Up to now, only two DME synthesis procedures from synthesis gas have been claimed, whereby one is the traditional methanol synthesis, followed by a dehydration step and the other is a direct conversion of synthesis gas to DME in one single step.

Recently, attention has been directed towards the direct synthesis of dimethyl ether from synthesis gas, using a catalytic system that combines a methanol synthesis catalyst and a catalyst for dehydration of said alcohol. It was confirmed on the basis of experimental studies that both, the stage of methanol synthesis and the stage of methanol dehydration, could be conducted simultaneously on one appropriate catalytic system. Depending upon the applied synthesis gas the catalyst might additionally show water gas shift activity.

Most known methods of producing methanol involve synthesis gas. Synthesis gas is a mixture of mainly hydrogen, carbon monoxide and carbon dioxide, whereby methanol is produced out of it over a catalyst.

CO+2H₂ [symbol] CH₃OH

In a following step methanol can be converted into DME by dehydration over an acidic catalyst.

2CH₃OH [symbol] CH₃OCH₃+H₂O

In the direct DME production there are mainly two overall reactions that occur from synthesis gas. These reactions, reaction (1) and reaction (2), are listed below.

3CO+3H₂ [symbol] CH₃OCH₃+CO₂ (1)

2CO+4H₂ [symbol] CH₃OCH₃+H₂O (2)

Reaction (1) occurs with the combination of three reactions, which are methanol synthesis reaction, methanol dehydration reaction, and water gas shift reaction:

2CO+4H₂ [symbol] 2CH₃OH (methanol synthesis reaction)

2CH₃OH [symbol] CH₃OCH₃+H₂O (methanol dehydration reaction)

CO+H₂O [symbol] CO₂+H₂ (water gas shift reaction)

The reaction (1) has a stoichiometric ratio H₂/CO of 1:1 and has some advantages over reaction (2). For example reaction (1) generally allows higher single pass conversions and less energy-consuming in comparison to the removal of water from the system in reaction (2).

Methods for the preparation of dimethyl ether are well-known from prior art. Several methods are described in the literature where DME is produced directly in combination with methanol by the use of a catalyst active body in both the synthesis of methanol from synthesis gas and methanol dehydration. Suitable catalysts for the use in the synthesis gas conversion stage include conventionally employed methanol catalyst such as copper and/or zinc and/or chromium-based catalyst and methanol dehydration catalyst.

The document U.S. Pat. No. 6,608,114 B1 describes a process for producing DME by dehydrating the effluent stream from the methanol reactor, where the methanol reactor is a slurry bubble column reactor (SBCR), containing a methanol synthesis catalyst that converts a synthesis gas stream comprising hydrogen and carbon monoxide into an effluent stream comprising methanol.

Document WO 2008/157682 A1 provides a method of forming dimethyl ether by bimolecular dehydration of methanol produced from a mixture of hydrogen and carbon dioxide, obtained by reforming methane, water, and carbon dioxide in a ratio of about 3 to 2 to 1. Subsequent use of water produced in the dehydration of methanol in the bi-reforming process leads to an overall ratio of carbon dioxide to methane of about 1:3 to produce dimethyl ether.

Document WO 2009/007113 A1 describes a process for the preparation of dimethyl ether by catalytic conversion of synthesis gas to dimethyl ether comprising contacting a stream of synthesis gas, comprising carbon dioxide with one or more catalysts active in the formation of methanol and the dehydration of methanol to dimethyl ether, to form a product mixture comprising the components dimethyl ether, carbon dioxide and unconverted synthesis gas, washing the product mixture comprising carbon dioxide and unconverted synthesis gas in a first scrubbing zone with a first solvent rich in dimethyl ether and subsequently washing the effluent from the first scrubbing zone in a second scrubbing zone with a second solvent rich in methanol to form a vapor stream comprising unconverted synthesis gas stream with reduced content of carbon dioxide transferring the vapor stream comprising unconverted synthesis gas stream with reduced carbon dioxide content for the further processing to dimethyl ether.

Document WO 2007/005126 A2 describes a process for the production of synthesis gas blends, which are suitable for conversion either into oxygenates such as methanol or into Fischer-Tropschliquids.

The U.S. Pat. No. 6,191,175 B1 describes an improved process for the production of methanol and dimethyl ether mixture rich in DME from essentially stoichiometrically balance synthesis gas by a novel combination of synthesis steps.

In document US 2008/125311 A1 is a catalyst used for producing dimethyl ether, a method of producing the same, and a method of producing dimethyl ether using the same. More particularly, the present invention relates to a catalyst used for producing dimethyl ether comprising a methanol synthesis catalyst produced by adding one or more promoters to a main catalyst comprised of a Cu—Zn—Al metal component and a dehydration catalyst formed by mixing Aluminum Phosphate (AlPO₄) with gamma alumina, a method of producing the same, and a method of producing dimethyl ether using the same, wherein a ratio of the main catalyst to the promoter in the methanol synthesis catalyst in a range of 99/1 to 95/5, and a mixing ratio of the methanol synthesis catalyst to the dehydration catalyst is in a range of 60/40 to 70/30.

The processes for the preparation of dimethyl ether according to the prior art bear the drawbacks that different steps have to be undergone to get an efficient DME production. Besides this, the catalyst used in the method known in prior art does not achieve the thermodynamic possibilities. Therefore it is still desirable to increase the yield of DME in the synthesis gas conversion.

### SUMMARY OF THE INVENTION

The object of the present invention is to provide a catalytically active body that shows the ability to convert CO-rich synthesis gas selectively into dimethyl ether and CO₂, whereby ideally the yield of the DME is increased in comparison to the state of the art. If the conversion is incomplete, the resulting off-gas comprises hydrogen and carbon monoxide preferably in the ratio H₂/CO˜1. Thus the off-gas can be recycled directly after the separation of the product DME and CO₂. In addition, it is an object of the present invention to provide a method for the preparation of a catalytically active body and a method for the preparation of dimethyl ether from synthesis gas, comprising the inventive catalytically active body and also the use of the catalytically active body for the preparation of dimethyl ether from synthesis gas.

These objects are achieved by a catalytically active body for the synthesis of dimethyl ether from synthesis gas, comprising a mixture of:
(A) 70-95% by weight of a methanol-active component, selected from the group consisting of copper oxide, aluminum oxide, zinc oxide, amorphous aluminum oxide, ternary oxide or mixtures thereof;
(B) 5-30% by weight of an acid component comprising a zeolitic material; and
(C) 0-10% by weight of at least one additive, whereby the sum of the components (A), (B) and (C) is in total 100% by weight;
   wherein component (B) is obtainable by a process comprising the steps of:
(b1) providing a mixture comprising one or more sources for SiO2 and/or Al₂O₃ and one or more alkenyltrialkylammonium cation R¹R²R³R⁴N⁺-containing compounds as structure directing agent, wherein R¹, R², and R³ independently from one another stand for alkyl; and R⁴ stands for alkylene; and
(b2) crystallizing the mixture obtained in step (b1) to obtain a zeolitic material.

All wt.-% values are reported on a calcined basis (i.e. free of water, organic and ammonium).

In a preferred embodiment of the catalytically active body the one or more sources for SiO₂ which can be used in step 031) comprises one or more compounds selected from the group consisting of fumed silica, silica hydrosols, reactive amorphous solid silica, silica gel, silicic acid, water glass, sodium metasilicate hydrate, sesquisilicate, disilicate, colloidal silica, pyrogenic silica, silicic acid esters, and mixtures of two or more thereof, preferably from the group consisting of fumed silica, silica hydrosols, reactive amorphous solid silica, silica gel, colloidal silica, pyrogenic silica, tetraalkoxysilanes, and mixtures of two or more thereof, particularly preferably from the group consisting of fumed silica, reactive amorphous solid silica, silica gel, pyrogenic silica, (C₁-C₃)tetraalkoxysilanes, and mixtures of two or more thereof, very particularly preferably from the group consisting of fumed silica, (C₁-C₂)-tetraalkoxysilanes, and mixtures of two or more thereof, and even most preferably the one or more sources for SiO₂ comprises fumed silica and/or tetraethoxysilane.

The one or more sources for Al₂O₃ which can be used in step (b1) comprises one or more compounds selected from the group consisting of alumina, aluminates, aluminum alcoholates, aluminum salts, and mixtures of two or more thereof, preferably from the group consisting of alumina, aluminum salts, aluminum alcoholates, and mixtures of two or more thereof, particularly preferably from the group consisting of alumina, AlO(OH), Al(OH)₃, aluminum halide, aluminum sulfate, aluminum phosphate, aluminum fluorosilicate, aluminum triisopropylate, and mixtures of two or more thereof, very particularly preferably from the group consisting of AlO(OH), Al(OH)₃, aluminum chloride, aluminum sulfate, aluminum phosphate, aluminum triisopropylate, and mixtures of two or more thereof, wherein even most preferably the one or more sources for Al₂O₃ comprises AlO(OH) and/or aluminum sulfate, preferably aluminum sulfate.

In a preferred embodiment of the catalytically active body the alkyl-residues R¹, R², and R³ of the alkenyltrialkylammonium cation of step (b1) independently from one another stand for (C₁-C₆)-alkyl, preferably for (C₂-C₄)-alkyl, particularly preferably for (C₂-C₃)-alkyl, very particularly preferably for branched or unbranched propyl, and even most preferably for n-propyl.

In a preferred embodiment of the catalytically active body the alkenyl-residue R⁴ of the alkenyltrialkylammonium cation of step (b1) stands for (C₂-C₆)-alkenyl, preferably for (C₂-C₄)-alkenyl, particularly preferably for (C₂-C₃)-alkenyl, very particularly preferably for 2-propen-1-yl, 1-propen-1-yl, or 1-propen-2-yl, and even most preferably 2-propen-1-yl or 1-propen-1-yl, and wherein even more preferably the mixture provided in step (b1) comprises two or more R¹R²R³R⁴N⁺-containing compounds, wherein R⁴ of the two or more compounds are different from one another and stand for (C₂-C₆)-alkenyl, preferably for (C₂-C₄)-alkenyl, particularly preferably for (C₂-C₃)-alkenyl, very particularly preferably for 2-propen-1-yl, 1-propen-1-yl, or 1-propen-2-yl, and even most preferably for 2-propen-1-yl and 1-propen-1-yl.

The structure directing agent provided in step (b1) comprises one or more compounds selected from the group consisting of N—(C₂-C₄)-alkenyl-tri-(C₂-C₄)-alkylammonium hydroxides, more preferably from the group consisting of N-(2-propen-1-yl)-tri-n-propylammonium hydroxide, N-(1-propen-1-yl)-tri-n-propylammonium hydroxide, N-(1-propen-2-yl)-tri-n-propylammonium hydroxide, and mixtures of two or more thereof.

In step (b1) according to the present invention, the mixture can be prepared by any conceivable means, wherein mixing by agitation is preferred, preferably by means of stirring.

In preferred embodiments of the inventive process, the mixture provided in step WO further comprises one or more solvents. According to the inventive process, there is no particular restriction whatsoever neither with respect to the type and/or number of the one or more solvents, nor with respect to the amount in which they may be used in the inventive process provided that a zeolitic material may be crystallized in step (b2). According to the inventive process it is however preferred that the one or more solvents comprise water, and more preferably distilled water, wherein according to particularly preferred embodiments distilled water is used as the only solvent in the mixture provided in step (b1).

The crystallization in step (b2) involves heating of the mixture at a temperature ranging from 90 to 210° C., preferably from 110 to 200° C., particularly preferably from 130 to 190° C., very particularly preferably from 145 to 180° C., and even most preferably from 155 to 170° C.

The crystallization in step (b2) is conducted under solvothermal conditions, meaning that the mixture is crystallized under autogenous pressure of the solvent which is used, for example by conducting heating in an autoclave or other crystallization vessel suited for generating solvothermal conditions. In particularly preferred embodiments wherein the solvent comprises water, preferably distilled water, heating in step (b2) is accordingly preferably conducted under hydrothermal conditions.

The apparatus which can be used in the present invention for crystallization is not particularly restricted, provided that the desired parameters for the crystallization process can be realized, in particular with respect to the preferred embodiments requiring particular crystallization conditions. In the preferred embodiments conducted under solvothermal conditions, any type of autoclave or digestion vessel can be used.

Furthermore, as regards the period in which the preferred heating in step (b2) of the inventive process is conducted for crystallizing the zeolitic material, there is again no particular restriction in this respect provided that the period of heating is suitable for achieving crystallization. Thus, by way of example, the period of heating may range anywhere from 5 to 120 h, wherein preferably heating is conducted from 8 to 80 h, more preferably from 10 to 50 h, and even more preferably from 13 to 35 h. According to particularly preferred embodiments heating in step (2) of the inventive process is conducted for a period of from 15 to 25 h.

According to preferred embodiments of the present invention, wherein the mixture is heated in step (b2), said heating may be conducted during the entire crystallization process or during only one or more portions thereof, provided that a zeolitic material is crystallized. Preferably, heating is conducted during the entire duration of crystallization.

Further regarding the means of crystallization in step (b2) of the inventive process, it is principally possible according to the present invention to perform said crystallization either under static conditions or by means of agitating the mixture. According to embodiments involving the agitation of the mixture, there is no particular restriction as to the means by which said agitation may be performed such that any one of vibrational means, rotation of the reaction vessel, and/or mechanical stirring of the reaction mixture may be employed to this effect wherein according to said embodiments it is preferred that agitation is achieved by stirring of the reaction mixture. According to alternatively preferred embodiments, however, crystallization is performed under static conditions, i.e. in the absence of any particular means of agitation during the crystallization process.

The process for the preparation of the acid component (B) further comprising one or more of the following steps of

(b3) isolating the zeolitic material, preferably by filtration, and/or
(b4) washing the zeolitic material, and/or
(b5) drying the zeolitic material, and/or
(b6) subjecting the zeolitic material to an ion-exchange procedure,
wherein—if necessary—in the at least one step (b6) one or more ionic non-framework elements contained in the zeolite framework are ion-exchanged against one or more cations and/or cationic elements, wherein the one or more cation and/or cationic elements are preferably selected from the group consisting of H⁺, NH₄⁺, Sr, Zr, Cr, Mo, Fe, Co, Ni, Cu, Zn, Ru, Rh, Pd, Ag, Os, Ir, Pt, Au, and mixtures of two or more thereof, particularly preferably from the group consisting of H⁺, NH₄⁺, Sr, Cr, Mo, Fe, Co, Ni, Cu, Zn, Ag, and mixtures of two or more thereof, very particularly preferably from the group consisting of H⁺, NH₄⁺, Cr, Mo, Fe, Ni, Cu, Zn, Ag, and mixtures of two or more thereof, and even most preferably from the group consisting of Mo, Fe, Ni, Cu, Zn, Ag, and mixtures of two or more thereof, wherein the one or more ionic non-framework elements preferably comprise H⁺ and/or an alkali metal, the alkali metal preferably being selected from the group consisting of Li, Na, K, Cs, and combinations of two or more thereof, particularly preferably from the group consisting of Li, Na, K, and combinations of two or more thereof, wherein very particularly preferably the alkali metal is Na and/or K, even most preferably Na.

The steps (b3), (b4), (b5) and/or (b6) can be conducted in any order, and wherein one or more of said steps is preferably repeated one or more times.

Isolation of the crystallized product can be achieved by any conceivable means. Preferably, isolation of the crystallized product can be achieved by means of filtration, ultrafiltration, diafiltration, centrifugation and/or decantation methods, wherein filtration methods can involve suction and/or pressure filtration steps. According to preferred embodiments, and in particular according to the particular and preferred embodiments of the present invention wherein one or more elements suitable for isomorphous substitution have been employed, it is preferred that the reaction mixture is adjusted to a pH comprised in the range of from 6 to 8, preferably from 6.5 to 7.5, and even more preferably of from 7 to 7.4 prior to isolation, Within the meaning of the present invention, pH values preferably refer to those values as determined via a standard glass electrode.

With respect to one or more optional washing procedures, any conceivable solvent can be used. Washing agents which may be used are, for example, water, alcohols, such as methanol, ethanol or propanol, or mixtures of two or more thereof. Examples of mixtures are mixtures of two or more alcohols, such as methanol and ethanol or methanol and propanol or ethanol and propanol or methanol and ethanol and propanol, or mixtures of water and at least one alcohol, such as water and methanol or water and ethanol or water and propanol or water and methanol and ethanol or water and methanol and propanol or water and ethanol and propanol or water and methanol and ethanol and propanol. Water or a mixture of water and at least one alcohol, preferably water and ethanol, is preferred, distilled water being very particularly preferred as the only washing agent.

Preferably, the separated zeolitic material is washed until the pH of the washing agent, preferably the washwater, is in the range of from 6 to 8, preferably from 6.5 to 7.5.

Drying procedures (b5) preferably include heating and/or applying vacuum to the zeolitic material. In envisaged embodiments of the present invention, one or more drying steps may involve spray drying, preferably spray granulation of the zeolitic material.

In embodiments which comprise at least one drying step, the drying temperatures are preferably in the range of from 25° C. to 150° C., more preferably of from 60 to 140° C., more preferably of from 70 to 130° C. and even more preferably in the range of from 75 to 125° C. The durations of drying are preferably in the range of from 2 to 60 h, more preferably in the range of 6 to 48 hours, more preferably of from 12 to 36 h, and even more preferably of from 18 to 30 h.

The BET surface area of the zeolitic material obtained by the previous described process and determined according to DIN 66135 ranges from 50 to 700 m²/g, preferably from 200 to 600 m²/g, particularly preferably from 350 to 500 m²/g, very particularly preferably from 390 to 470 m²/g, and even most preferably from 420 to 440 m²/g.

The synthetic zeolitic material (B) having an MFI-type framework structure comprising SiO2 and Al₂O₃, wherein said material having an X-ray diffraction pattern comprising at least the following reflections:

| | Diffraction angle 2θ/° |
|---|---|
| Intensity (%) | [Cu K(alpha 1)] |
| 15-55 | 7.88-8.16 |
| 11-35 | 8.83-9.13 |
| 100 | 23.04-23.46 |
| 27-40 | 23.68-23.93 |
| 21-66 | 23.85-24.23 |
| 22-44 | 24.29-24.71 |

wherein 100% relates to the intensity of the maximum peak in the X-ray powder diffraction pattern. The zeolitic material displaying the aforementioned X-ray diffraction pattern comprises ZSM-5. The SiO₂:Al₂O₃ molar ratio of the zeolitic material (B) may range from 0.5 to 500, preferably from 1 to 400, more preferably from 5 to 300, more preferably from 20 to 200, more preferably from 30 to 150, more preferably from 30 to 120, and even most preferably from 40 to 100.

In a preferred embodiment of the catalytically active body the mixture comprises:
(A) 70-95% by weight of a methanol-active component, selected from the group consisting of copper oxide, aluminum oxide, zinc oxide, amorphous aluminum oxide, ternary oxide or mixtures thereof, wherein the component (A) has a particle size distribution characterized by a D-10 value of 3-140 μm, a D-50 value of 20-300 μm, and a D-90 value of 180-900 μm,
(B) 5-30% by weight of an acid component comprising a zeolitic material as defined above, wherein the component (B) has a particle size distribution characterized by a D-10 value of 3-140 μm, a D-50 value of 20-300 μm, and a D-90 value of 180-900 μm,
(C) 0-10% by weight of a at least one additive, wherein the sum of the components (A), (B), and

(C) is in total 100% by weight and the particle size of components (A) and (B) is maintained in the catalytically active body.

This particle size distribution can be determined via state of the art analysis techniques, e.g. via analysis apparatus like Mastersizer 2000 or 3000 by Malvern Instruments GmbH. The particle size distribution in the sense of the invention is characterized by the D10-, D50-, and D-90 value. The definition of D10 is: that equivalent diameter where 10 mass % (of the particles) of the sample has a smaller diameter and hence the remaining 90% is coarser. The definition of D50 and D90 can be derived similarly (see: HORIBA Scientific, A Guidebook to Particle Size Analysis” page 6).

Preferably, the components (A) or (B) have a particle size distribution characterized by a D-10, D50, and D-90 value of 3-140 μm, 20-300 μm, and 180-900 μm respectively. In a further embodiment the particle size distribution from component (A) can be different from component (B) and (C).

In the sense of the present invention a catalytically active body can be a body known in the art that contains pores or channels or other features for enlargement of surface, which will help to bring the educts in contact that they can react to the desired product. A catalytically active body in the sense of the present invention can be understood as a physical mixture, whereby the components (A) and (B) contact each other and presenting channels and/or pores between their contact surfaces. Preferably, the components (A) and (B) are not melted or sintered at their contact surfaces.

A methanol-active component in the sense of the present invention is a component which leads to the formation of methanol, starting from hydrogen, carbon monoxide or carbon dioxide or mixtures thereof. Preferably, the methanol-active compound is a mixture of copper oxide, aluminum oxide and zinc oxide, whereby copper oxide can consist of all kinds of oxides of copper. In particular, copper has the oxidation state (I) or (II) in the oxide. The aluminum oxide according to the present invention can also be referred to γ-alumina or corundum, whereby zinc in zinc oxide in the sense of the present invention preferably has the oxidation state (II).

In a preferred embodiment of the catalytically active body, the component (A) comprises 50-80% by weight of copper oxide, 15-35% by weight of ternary oxide and 15-35% by weight of zinc oxide and the sum of which is in total 100% by weight. In particular the component (A) comprises 65-75% by weight of copper oxide, 20-30% by weight of ternary oxide and 20-30% by weight of zinc oxide and the sum of which is in total 100% by weight.

Preferably, the ternary oxide of component (A) is a zinc-aluminum-spinel.

In a preferred embodiment of the catalytically active body, the component (A) comprises 50-80% by weight of copper oxide, 2-8% by weight of boehmite and 15-35% by weight of zinc oxide and the sum of which is in total 100% by weight. In particular the component (A) comprises 65-75% by weight of copper oxide, 3-6% by weight of boehmite and 20-30% by weight of zinc oxide and the sum of which is in total 100% by weight.

In a preferred embodiment of the catalytically active body, the component (A) comprises 50-80% by weight of copper oxide, 2-8% by weight of amorphous aluminum oxide and 15-35% by weight of zinc oxide and the sum of which is in total 100% by weight. In particular the component (A) comprises 65-75% by weight of copper oxide, 3-6% by weight of amorphous aluminum oxide and 20-30% by weight of zinc oxide and the sum of which is in total 100% by weight.

In a preferred embodiment of the catalytically active body, the component (A) comprises 50-80% by weight of copper oxide, 2-8% by weight of aluminum oxide and 15-35% by weight of zinc oxide and the sum of which is in total 100% by weight. In particular the component (A) comprises 65-75% by weight of copper oxide, 3-6% by weight of aluminum oxide and 20-30% by weight of zinc oxide and the sum of which is in total 100% by weight.

In the sense of the present invention an additive (C) can be a structure-promoter like but not limited a thermally decomposable compound like polymers, wood dust, flour, graphite, film material, a painting, straw, strearic acid, palmitic acid, celluloses or a combination thereof. For example, the structure-promotor can help to build up pores or channels.

In a preferred embodiment the catalytically active body consists of 70-95% by weight of the methanol-active component (A) and 5-30% by weight of the acid component (B) and the sum of (A) and (B) being in total 100% by weight. Preferably the catalytically active body consists of 75-85% by weight of the methanol-active component (A) and 15-25% by weight of the acid component (B) and the sum of (A) and (B) being in total 100% by weight. One advantage of this composition is that the turnover of the reaction of the methanol-active compound (A) and the acid compound (B) is favored, because the highly integrated catalyst system combines the methanol synthesis, water gas shift activity, and methanol dehydration catalyst in a close proximity. Therefore an optimum efficiency can be obtained.

In a preferred embodiment the catalytically active body is a pellet with a size in the range from 1×1 mm to 10×10 mm, preferably in the range from 2×2 mm to 7×7 mm. The pellet is obtained by pressing the mixture of the components (A), (B) and (C) to a pellet. In the sense of the present invention a pellet can be obtained by pressing the components (A), (B) and optionally (C) under force to the pellet, whereby the shape of the pellet can be ring-shaped, star-shaped or spherical-shaped. Furthermore the pellet can be hollow strings, triloops, multihole pellets, extrudates and alike.

The present invention further relates to a method for the preparation of a catalytically active body, comprising the step:
c) preparation a physical mixture comprising:
(A) 70-95% by weight of a methanol-active component, selected from the group consisting of copper oxide, aluminum oxide, zinc oxide, amorphous aluminum oxide, ternary oxide or mixtures thereof;
(B) 5-30% by weight of an acid component comprising a zeolitic material, obtainable by a process comprising the steps b1) and b2) already defined above; and
(C) 0-10% by weight of a at least one additive, whereby the sum of the components (A), (B) and (C) is in total 100% by weight.

In this context, the meanings of the features are the same as for the catalytically active body already mentioned.

In the sense of the present invention preparing a physical mixture means that the different compounds (A), (B) and (C) are brought in contact without further chemical modification.

In a preferred embodiment of the method, the component (A) has a particle size distribution characterized by a D-10 value of 3-140 μm, a D-50 value of 20-300 μm, and a D-90 value of 180-900 μm, whereby the component (B) has a particle size distribution characterized by a D-10 value of 3-140 μm, a D-50 value of 20-300 μm, and a D-90 value of 180-900 μm and the particle size distribution of components (A) and (B) is maintained in the catalytically active body. In a preferred embodiment the method comprising further the steps:
a) precipitation a copper-, zinc-, or aluminumsalt or a mixture thereof,
b) calcination of the product obtained in step a).

Preferably, the steps a) and b) are carried out before the step c). Preferably, the obtained product consists after step b) of 70-95% by weight of a methanol-active component (A), selected from the group consisting of copper oxide, aluminum oxide and zinc oxide or mixtures thereof, 5-30% by weight of an acid component (B), selected from the group consisting of alumosilicate, γ-alumina and zeolite or mixtures thereof. Preferably, after step c) the component (A) has a particle size distribution characterized by a D-10 value of 3-140 μm, a D-50 value of 20-300 μm, and a D-90 value of 180-900 μm and the component (B) has a particle size distribution characterized by a D-10 value of 3-140 μm, a D-50 value of 20-300 μm, and a D-90 value of 180-900 μm.

Preferably, the method comprises at least spray drying, filtration, grinding, sieving or further steps, known in the art to create a catalytically active body, or combinations thereof.

In the sense of the present invention precipitation is a method for the formation of a solid in a solution or inside another solid during a chemical reaction or by diffusion in a solid. The precipitation techniques are known in the art, see also Ertl, Gerhard, Knözinger, Helmut, Schüth, Ferdi, Weitkamp, Jens (Hrsg.) “Handbook of Heterogeneous Catalysis” 2nd edition 2008, Wiley VCH Weinheim, Vol. 1, chapter 2. For example salts of copper, zinc or aluminum are dissolved in a solvent, in particular water. At least two of the salts of either copper, zinc, or aluminum can be heated and a basic solution can be prepared and added. Both solutions can be added in parallel to the template, till the salt-solution is consumed. After this the suspension is vacuumed, dried, and calcinated under air flow.

Preferred anions in the salts for copper, zinc, or aluminum are selected from the group consisting of nitrate, acetate, halide, carbonate, nitrite, sulfate, sulfite, sulfide, phosphate ion or silicate. In particular, salts of copper, zinc or aluminum formed with the above mentioned anions can be converted into oxides of copper, zinc or aluminum applying a calcination step.

Calcination in the sense of the present invention can be understood as a thermal treatment process applied to ores and other solid materials to bring about a thermal decomposition, phase transition, or removal of a volatile fraction. The calcination process normally takes place at temperatures below the melting point of the product materials. Mostly it is done under oxygen-containing atmosphere. In some cases the calcination can be performed under inert atmosphere (e.g. nitrogen). Calcination is to be distinguished from roasting, in which more complex gas-solid reactions take place between the furnace atmosphere and the solids.

In particular the components (A), (B) and (C) can be compacted in a presser, a squeezer, a crusher or a squeezing machine, preferably after step a), b) or c). Compacting in the sense of the present invention can mean that particles of a defined particle size distribution are pressed to bodies, which have a diameter in the range of 1 to 10 mm and a height of 1 to 10 mm. Preferably the particle size distribution is still left after the compacting.

In a preferred embodiment of the method a pellet is formed, preferably with a size in the range from 1×1 mm to 10×10 mm, especially in the range from 2×2 mm to 7×7 mm.

In a preferred embodiment of the method, the components (A) and (B) are independently pressed through at least one sieve, whereby the sieve exhibits a mesh size from 0.005 to 1.5 mm in order to obtain a particle size distribution characterized by a D-10 value of 3-140 μm, a D-50 value of 20-300 μm, and a D-90 value of 180-900 μm. Preferably the sieve exhibits a mesh size from 0.005 to 0.90 mm and in particular a mesh size from 0.005 to 0.80 mm. In particular the particles can also exhibit particle size distribution characterized by a D-10, D-50, and D-90 value of 3-140 μm, 20-300 μm, and 180-900 μm respectively. Thereby the components (A) and (B) can be obtained as particles with a defined particle size distribution, also referred in the sense of the present invention as a split-fraction. Because of this split-fraction the CO-conversion increases when synthesis gas contacts the split-fraction. Furthermore the yield of the DME increases, when synthesis gas is converted to DME by the catalytically active body. Preferably, this step is included in step c).

In a further embodiment component (C) is admixed to the components (A) and (B) before sieving.

In a preferred embodiment of the preparation of a catalytically active body at least three different sieves are used, whereby the components (A) and (B) are pressed in direction from the sieve with the biggest mesh size to the sieve with the smallest mesh size. By using three sieves with different mesh sizes the components (A) and (B) are initially pressed into the sieve with the biggest mesh size, which results in particles with the maximal size of the mesh size of this sieve. Preferably, the particle size distribution of the components (A) and (B) is characterized by a D-10 value of 3-140 μm, a D-50 value of 20-300 μm, and a D-90 value of 180-900 μm. These particles can also be broken during the first sieving, so that smaller particles are obtained, which can go through the second sieve, which exhibits a smaller mesh size. Therefore a first fraction with a specific particle size distribution can be obtained before the second sieve. This fraction can also be used as a catalytically active body. Besides this, the particles which go through the second sieve with a mesh size smaller than the first sieve, but bigger than the third sieve, can be obtained behind the second sieve and before the smallest sieve with the smallest mesh size. Also here the particles obtained after the second (middle) sieve can be used as a catalytically active body. In addition to this, the particles obtained after the sieve with the biggest mesh size could be pressed through the second sieve in order to reduce the particle size.

In a preferred embodiment of the method according to the present invention in step a) a part of the component (A) is prepared by precipitation reaction and/or calcination. In the sense of the present invention precursors of the component (A) in form of a salt in a solution can be heated and adjusted to a defined pH-value. After this, a calcination step can be carried out, whereby calcination is known from prior art. These steps can lead to the desired component (A).

In a preferred embodiment of the inventive method at least one part of component (A) is precipitated and whereby at least another part of component (A), which is not subjected to the first precipitation, is added to the precipitate. Preferably, it is added by spray drying or precipitation.

In a preferred embodiment of the inventive method, the method further comprises the step d) adding a mixture of hydrogen and nitrogen to component (A) and/or (B). Preferably the content of the volume of the hydrogen is less than 5% in the mixture.

The present invention further relates to a method for the preparation of dimethyl ether from synthesis gas comprising at least the steps:
e) reducing the catalytically active body
f) contacting the catalytically active body in a reduced form with hydrogen and at least one of carbon monoxide or carbon dioxide.

In a further embodiment the method comprising the steps:
g) providing the inventive catalytically active body, in particular in form of pellets
h) disposing the catalytically active body in a reactor,
i) reducing the catalytically active body at a temperature between 140° C. and 240° C. with at least a nitrogen and hydrogen mixture.

The present invention further relates to the use of a catalytically active body according to the present invention for the preparation of dimethyl ether. Preferred admixtures and preferred methods for the preparation are mentioned above and also included in the use.

The inventive catalytically active body is characterized by a high turnover of carbon monoxide, preferably at 180° C. to 350° C. and particularly preferably at 200° C. to 300° C. For example, a suitable pressure for the synthesis of DME is preferably in the range from 20 to 80 bar and particularly preferably from 30 to 50 bar.

The present invention is further illustrated by the following examples:

### EXAMPLE 1

### Synthesis of Inventive Catalyst (Cat I)

1a—Synthesis of the Methanol-Active Component (A1)

### I. Precipitation:

A sodium bicarbonate solution (20%) was prepared by dissolving 11 kg sodium bicarbonate in 44 kg demineralised water. Also a Zn/Al-solution was prepared by dissolving 6.88 kg zinc nitrate and 5.67 kg aluminum nitrate in 23.04 kg water. Both solutions were heated to 70° C. and combined via a pump device in a precipitation pot filled with 12.1 L warm demineralised water at 70° C. and the pH was adjusted at a pH=7. After precipitation was completed, the mixture was further stirred for 15 hours and the resulting suspension was filtered through a vacuum filter and washed nitrate-free with water. The product was dried for 24 h at 120° C. and calcined for 1 h at 350° C. under air flow.

### II. Precipitation:

A sodium bicarbonate solution (20%) was prepared by dissolving 25 kg sodium bicarbonate in 100 kg demineralised water. Also a Cu/Zn-nitrate solution was prepared by dissolving 26.87 kg copper nitrate and 5.43 kg zinc nitrate in 39 kg water. Both solutions were heated to 70° C. After the Cu/Zn-nitrate solution had reached a temperature of 70° C., the product of the precipitation I was slowly added to this solution and the pH-value was adjusted to pH=2 by an aqueous solution of nitric acid (65%). Both solutions (sodium bicarbonate and Cu/Zn-nitrate solution) were combined via a pump device in a precipitation pot filled with 40.8 L demineralised water at 70° C. and the pH was adjusted at a pH=6.7. After precipitation was completed, the mixture was further stirred for 10 hours whereby the pH-value was adjusted to pH=6.7 with the nitric acid (65%) and the resulting suspension was filtered through a vacuum filter and washed nitrate-free with water. The product was dried for 72 h at 120° C. and calcined for 3 h at 300° C. under air flow. After cooling to room temperature the methanol-active compound (A1) containing 70 wt.-% CuO, 5.5 wt.-% Al₂O₃ and 24.5 wt.-% ZnO was ready for use. The corresponding D-10. D-50 and D-90 values are listed in Table 2.

1 b—Synthesis of the Acid Component (B1)

Synthesis of a MFI structured zeolite being crystallized by means of the structure directing agent N-allyl-tri-propylammonium hydroxide (ATPAOH):

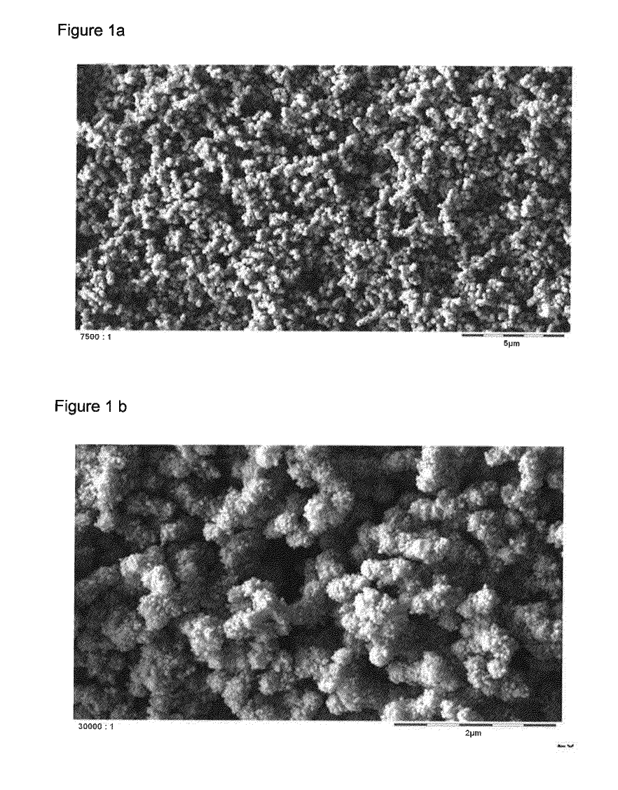

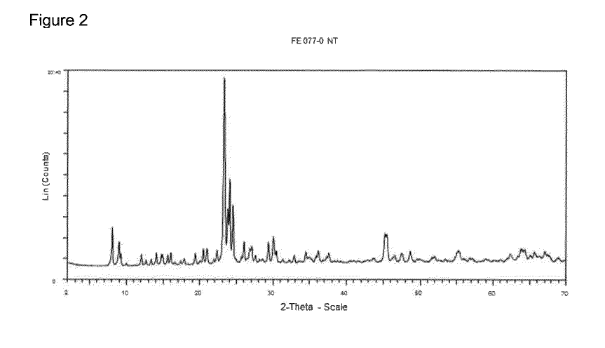

A mixture of 40 wt.-% ATPAOH in H₂O (333 ml) was stirred with tetraethylorthosilicate (757 g) and distilled H₂O (470 g) for 60 min at room temperature. Afterwards 746 g of ethanol were removed at 95° C. from the reaction gel by distillation. After cooling down, 746 g H₂O as well as Al₂(SO₄)₃*18 H₂O (24.3 g) dissolved in 20 ml distilled H₂O were added. The dispersion was transferred into a 2.5 L autoclave, which was then heated to 155° C. for 24 h. After cooling down to room temperature, the formed solid was filtered, repeatedly washed with distilled water and dried at 120° C. for 16 h. 210 g of a white powder was received. The organic residuals were removed by calcination at 500° C. for 6 h. The characterization of the obtained white powder by means of XRD, N₂—Sorption and Ar-Sorption showed a pure MFI structured material (=B1) with a an average crystal size of 83 nm+/−20 nm, a surface area of 407 m²/g (BET), a pore volume of 0.190 cm³/g and a median pore width of 0.59 nm. The elemental analysis showed 41 wt.-% Si, 0.76 wt.-% Al and <0.01 wt.-% Na in the sample. By means of SEM and XRD no other side phases could be observed in the product (see FIGS. 1a, 1b and 2). The corresponding D-10. D-50 and D-90 values are listed in Table 2.

1c—Preparation of the Final Catalytically Active Body

The methanol-active component (A1) and the acid component (B1) were compacted separately in a tablet press. The obtained molding (diameter=ca, 25 mm, height=ca, 2 mm) was squeezed through sieves with an appropriate mesh size, so that the desired split fraction was obtained. From both fractions the proper quantity was weight in (9/1, 8/2, or 7/3 methanol-active/acidic component) and mixed with the other component in a mixing machine (Heidolph Reax 2 or Reax 20/12) to obtain Cat I in the form of split.

### EXAMPLE 2

### Synthesis of Comparative Catalyst (Cat II)

2a—Synthesis of the Methanol-Active Component (A2)

Component (A2) was identical to the methanol-active component (A1) as described in Example 1a,

2b—Acid Component (B2)

Acid component (B2) was a commercially obtainable ZSM-5 zeolite powder [(ZEOcat® PZ-2/100 (Zeochem, Switzerland)] having the following composition:

44 wt.-% Si, 0.84 wt.-% Al and 0.02 wt.-% Na. The corresponding D-10, D-50 and D-90 values are listed in Table 2.
2c—Preparation of the Final Catalytically Active Body

The methanol-active component (A2) and the acid component (B2) were compacted separately in a tablet press. The obtained molding (diameter=ca, 25 mm, height=ca, 2 mm) was squeezed through sieves with an appropriate mesh size, so that the desired split fraction (0.15-0.2 mm) was obtained. From both fractions the proper quantity was weight in (9/1, 8/2, or 7/3 methanol-active/acidic component) and mixed with the other component in a mixing machine (Heidolph Reax 2 or Reax 20/12) to obtain Cat II in the form of split.

### EXAMPLE 3

### Testing Conditions for Final Catalytically Active Body in the Form of Split

The catalytically active body (5 cm³ by volume) was incorporated in a tubular reactor (inner diameter 4 cm, bedded in a metal heating body) on a catalyst bed support consisting of alumina powder as layer of inert material and was pressure-less reduced with a mixture of 1 Vol.-% H₂ and 99 Vol.-% N₂. The temperature was increased in intervals of 8 h from 150° C. to 170° C. and from 170° C. to 190° C. and finally to 230° C. At a temperature of 230° C. the synthesis gas was introduced and heated within 2 h up to 250° C. The synthesis gas consisted of 45% H₂ and 45% CO and 10% inert gas (argon). The catalytically active body was run at an input temperature of 250° C., GHSV of 2400 h⁻¹ and a pressure of 50 bar.

### EXAMPLE 4

### Testing Conditions for Final Catalytically Active Body in the Form of Pellets

Tests for pelletized materials were conducted in a similar test rick compared to the setup described above for non-pelletized materials using the same routine. Only the geometry of the tubular reactor was modified (inner diameter of 3 cm instead of 4 cm). Tests for pelletized materials were done with a catalyst volume of 100 cm³.

### Results:

In the following Table 1 the results are presented. The comparative catalyst Cat II shows a lower turnover, whereby the inventive catalyst Cat I shows an increased value. Surprisingly the mixture of inventive material shows a significantly increased CO-conversion compared to Cat II. With respect to the selectivity patterns it is worth to mention that within the DME forming samples an equal selectivity of DME and CO₂ can be observed. This shows that all catalysts have a sufficient water gas shift activity that is needed to convert the water generated in the methanol dehydration step with CO into CO₂. Furthermore all catalysts show an adequate MeOH dehydration capability. This can be seen in the MeOH contents in the product streams in Table 1.

Inventive catalyst Cat I further shows a significant lower MeOH rate compared to Cat II. This shows that the acid component (B1) has a significant higher capability to convert MeOH into DME than the state of the art material (B2) (ZEOcat® PZ-2/100, ZSM5-100H).

Inventive catalyst Cat I in form of pellets reveals that the superior performance of Cat I compared to Cat II remains after the material was pelletized. Cat I (as pellet) also shows higher CO-conversions and a lower Methanol selectivity compared to Cat II (as pellet).

TABLE 1

| | CO- | S | S | S | S |
|---|---|---|---|---|---|
| | conversion | (MeOH) | (DME) | (CO2) | (Others) |
| | [%] | [vol. %] | [vol. %] | [vol. %] | [vol. %] |
| Cat I: split | 90.07 | 2.80 | 48.63 | 48.45 | 0.12 |
| (0.15-0.2 mm) | | | | | |
| A1/B1 (4/1) | | | | | |
| Cat II: split | 82.91 | 4.42 | 47.47 | 47.68 | 0.43 |
| (0.15-0.2 mm) | | | | | |
| A2/B2 (4/1) | | | | | |
| Cat I: Pellet | 85.34 | 1.55 | 48.89 | 49.05 | 0.51 |
| (3 × 3 mm) | | | | | |
| Cat II: Pellet | 73.46 | 3.96 | 47.57 | 48.20 | 0.27 |
| (3 × 3 mm) | | | | | |

All gaseous streams were analyzed via online-GC. Argon was used as internal standard to correlate in and off gas streams.

CO conversion was given as follows:

(COᵢₙ−(COₒᵤₜ*Argonᵢₙ/Argonₒᵤₜ))*100%

S(MeOH)=Volume (MeOH) in product stream/Volume (MeOH+DME+CO₂+Others without hydrogen and CO) in product stream*100%

S(DME)=Volume (DME) in product stream/Volume (MeOH+DME+CO₂+Others without hydrogen and CO) in product stream*100%

S(CO₂)=Volume (CO₂) in product stream/Volume (MeOH+DME+CO₂+Others without hydrogen and CO) in product stream*100%

S(Others)=Volume (Others) in product stream/Volume (MeOH+DME+CO₂+Others without hydrogen and CO) in product stream*100%

“Others” are compounds that are formed out of H₂ and CO in the reactor that are not MeOH, DME, or CO₂.

TABLE 2
Particle size distribution of components A1/A2, B1 and B2

| | D-10 [μm] | D-50 [μm] | D-90 [μm] |
|---|---|---|---|
| (A1)/(A2) | 5.42 | 146.57 | 389.14 |
| (B1) | 21.97 | 251.91 | 382.17 |
| (B2) | 3.47 | 200.82 | 334.78 |

1-23. (canceled)

24. A catalytically active body for synthesis of dimethyl ether from synthesis gas, comprising a mixture of:
(A) 70-95% by weight of a methanol-active component, selected from the group consisting of copper oxide, aluminum oxide, zinc oxide, amorphous aluminum oxide, ternary oxide or mixtures thereof;
(B) 5-30% by weight of an acid component comprising a zeolitic material; and
(C) 0-10% by weight of at least one additive, whereby the sum of the components (A), (B) and (C) is in total 100% by weight;
wherein component (B) is obtainable by a process comprising the steps of:
b1) providing a mixture comprising one or more sources for SiO₂ and/or Al₂O₃ and one or more alkenyltrialkylammonium cation R¹R²R³R⁴N⁺-containing compounds as structure directing agent, wherein R¹, R², and R³ independently from one another stand for alkyl; and R⁴ stands for alkylene; and
b2) crystallizing the mixture obtained in step (b1) to obtain a zeolitic material.

25. The catalytically active body according to claim 24, wherein R¹, R², and R³ of the structure directing agent independently from one another stand for (C₁-C₆)-alkyl, and R⁴ stands for (C₂-C₆)-alkenyl.

26. The catalytically active body according to claim 25, wherein R¹, R², and R³ of the structure directing agent independently from one another stand for branched or unbranched propyl, and R⁴ stands for 2-propen-1-yl or 1-propen-1-yl.

27. The catalytically active body according to claim 26, wherein the structure directing agent provided in step (b1) comprises N-(2-propen-1-yl)-tri-n-propylammonium hydroxide, N-(1-propen-1-yl)-tri-n-propylammonium hydroxide or N-(1-propen-2-yl)-tri-n-propylammonium hydroxide, or mixtures of two or more thereof.

28. The catalytically active body according to claim 24, wherein the component (A) has a particle size distribution characterized by a D-10 value of 3-140 μm, a D-50 value of 20-300 μm, and a D-90 value of 180-900 μm, wherein the component (B) has a particle size distribution characterized by a D-10 value of 3-140 μm, a D-50 value of 20-300 μm, and a D-90 value of 180-900 μm and the particle size distributions of the components (A) and (B) is maintained in the catalytically active body.

29. The catalytically active body according to claim 24, characterized in that component (A) comprises 50-80% by weight of copper oxide, 15-35% by weight of ternary oxide and 15-35% by weight of zinc oxide and the sum of which is in total 100% by weight.

30. The catalytically active body according to claim 24, characterized in that component (A) comprises 50-80% by weight of copper oxide, 2-8% by weight of boehmite and 15-35% by weight of zinc oxide and the sum of which is in total 100% by weight.

31. The catalytically active body according to claim 24, characterized in that component (A) comprises 50-80% by weight of copper oxide, 2-8% by weight of amorphous aluminum oxide and 15-35% by weight of zinc oxide and the sum of which is in total 100% by weight.

32. The catalytically active body according to claim 24, characterized in that component (A) comprises 50-80% by weight of copper oxide, 2-8% by weight of aluminum oxide and 15-35% by weight of zinc oxide and the sum of which is in total 100% by weight.

33. The catalytically active body according to claim 24, wherein the component (B) comprises 35-55% by weight of silicon, 0.15-4% by weight of aluminum, 45-65% by weight of oxygen and less than 0.01% by weight of sodium and the sum of which is in total 100% by weight.

34. The catalytically active body according to claim 24, wherein the catalytically active body consists of (A) 70-95% by weight of a methanol-active component and 5-30% by weight of a zeolitic material (B) and the sum of (A) and (B) being in total 100% by weight.

35. The catalytically active body according to claim 24, wherein the catalytically active body is a pellet with a size from 1×1 mm to 10×10 mm.

36. A method for preparing a catalytically active body, comprising the step:
c) preparation of a physical mixture comprising:
(A) 70-95% by weight of a methanol-active component, selected from the group consisting of copper oxide, aluminum oxide, zinc oxide, amorphous aluminum oxide, ternary oxide or mixtures thereof;
(B) 5-30% by weight of an acid component comprising a zeolitic material, obtainable by a process comprising the steps b1) and b2) as defined in claim 24; and
(C) 0-10% by weight of at least one additive, whereby the sum of the components (A), (B) and (C) is in total 100% by weight.

37. The method for preparing a catalytically active body according to claim 36, whereby the component (A) has a particle size distribution characterized by a D-10 value of 3-140 μm, a D-50 value of 20-300 μm, and a D-90 value of 180-900 μm, whereby the component (B) has a particle size distribution characterized by a D-10 value of 3-140 μm, a D-50 value of 20-300 μm, and a D-90 value of 180-900 μm and the particle size distribution of the components (A) and (B) is maintained in the catalytically active body.

38. The method for preparing a catalytically active body according to claim 36, comprising further the steps:
a) precipitating a copper-, zinc,- or aluminum salt or a mixture thereof to form a product,
b) calcining the product obtained in step a).

39. The method for preparing a catalytically active body according to claim 37, wherein a pellet is formed.

40. The method for preparing a catalytically active body according to claim 36, wherein the components (A) and (B) are independently pressed through at least one sieve exhibiting a mesh size from 0.005 to 1.5 mm in order to obtain a particle size distribution characterized by a D-10 value of 3-140 μm, a D-50 value of 20-300 μm, and a D-90 value of 180-900 μm.

41. The method for preparing a catalytically active body according to claim 36, wherein at least three different sieves are used, whereby the components (A) and (B) are pressed in direction from the sieve with the biggest mesh size to the sieve with the smallest mesh size.

42. The method for preparing a catalytically active body according to claim 36, wherein in step a) at least a part of the component (A) is prepared by precipitation reaction and/or calcination.

43. The method for preparing a catalytically active body according to claim 36, whereby at least one part of component (A) is precipitated to form a precipitate and whereby at least another part of component (A), which is not subjected to the first precipitation, is added to the precipitate.

44. The method for preparing a catalytically active body according to claim 36, wherein the method further comprises the step d) adding a mixture of hydrogen and nitrogen to component (A) and/or (B).

45. A method for preparing dimethyl ether from synthesis gas comprising at least the steps:
e) reducing the catalytically active body
f) contacting the catalytically active body in a reduced form with hydrogen and at least one of carbon monoxide or carbon dioxide.

46. Use of a catalytically active body according to claim 24 or obtained by a method according to claim 36 for the preparation of dimethyl ether.
